(19) United States
(12) Patent Application Publication
(10) Pub. No.: US 2024/0091517 A1
(43) Pub. Date: Mar. 21, 2024

(54) MEDICAL DEVICE FOR ULTRASOUND-ASSISTED DRUG DELIVERY

(71) Applicant: BOSTON SCIENTIFIC SCIMED, INC., Maple Grove, MN (US)

(72) Inventors: Misty L. Noble-Vranish, Bothell, WA (US); Sheeny Kate Lan Levengood, Bothell, WA (US); Eric Charles Whittaker, Edmonds, WA (US); Shuxian Song, Shoreline, WA (US); Curtis Cornell Genstler, Snohomish, WA (US); Christy Katherine Holland, Cincinnati, OH (US)

(73) Assignee: BOSTON SCIENTIFIC SCIMED, INC., Maple Grove, MN (US)

(21) Appl. No.: 18/368,805

(22) Filed: Sep. 15, 2023

Related U.S. Application Data
(60) Provisional application No. 63/406,913, filed on Sep. 15, 2022.

(51) Int. Cl. A61M 37/00; A61M 25/00

(52) U.S. Cl. CPC A61M 37/0092; A61M 25/003; A61M 2205/3606; A61M 2205/366

(57) ABSTRACT

A system for treating a vascular region includes an elongate catheter shaft having a distal end region, with an inner lumen formed in the elongate shaft and a fluid delivery lumen formed in the elongate shaft adjacent to the inner lumen. A treatment core is disposable within the inner lumen, and has one or more ultrasound transducers disposable adjacent to the distal end region of the elongate catheter shaft, the one or more ultrasound transducers including a proximal-most ultrasound transducer. The fluid delivery lumen is adapted such that fluid passing through the fluid delivery lumen exits the fluid delivery lumen at a position proximal to the proximal-most ultrasound transducer.

### CROSS-REFERENCE TO RELATED APPLICATIONS

This application claims the benefit of priority under 35 U.S.C. § 119 of U.S. Provisional Application No. 63/406,913, filed Sep. 15, 2022, the entire disclosure of which is hereby incorporated by reference.

### TECHNICAL FIELD

The present disclosure pertains to medical devices, and methods for manufacturing medical devices. More particularly, the present disclosure pertains to elongated intracorporeal medical devices including a tubular member connected with other structures, and methods for manufacturing and using such devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices. Moreover, there is a need for medical devices that can successfully infuse cavitation nuclei which enable sustained cavitation in an applied ultrasound filed. There is a need for ways to promote temporally stable bubble activity.

### BRIEF SUMMARY

This disclosure provides design, material, manufacturing method, and use alternatives for medical devices. An example may be found in a system for treating a vascular region. The system includes an elongate catheter shaft having a distal end region, with an inner lumen formed in the elongate catheter shaft and a fluid delivery lumen formed in the elongate catheter shaft adjacent to the inner lumen. A treatment core is disposed within the inner lumen, the treatment core including one or more ultrasound transducers disposed adjacent to the distal end region of the elongate catheter shaft, the one or more ultrasound transducers including a proximal-most ultrasound transducer. The fluid delivery lumen is adapted such that fluid passing through the fluid delivery lumen exits the fluid delivery lumen at a position proximal to the proximal-most ultrasound transducer.

Alternatively or additionally to any of the embodiments above, the inner lumen may have a non-circular cross-sectional shape.

Alternatively or additionally to any of the embodiments above, the inner lumen may have a central treatment region and one or more coolant regions disposed about the central treatment region.

Alternatively or additionally to any of the embodiments above, the fluid delivery lumen may include an opening that is formed in a side wall of the elongate catheter shaft.

Alternatively or additionally to any of the embodiments above, the opening may be a side-facing opening.

Alternatively or additionally to any of the embodiments above, the opening may be an end-facing opening.

Alternatively or additionally to any of the embodiments above, the opening may have a non-circular shape.

Alternatively or additionally to any of the embodiments above, the opening may have an oval shape.

Alternatively or additionally to any of the embodiments above, the system may further include one or more additional fluid delivery lumens that are formed in the elongate catheter shaft and are positioned adjacent to the inner lumen.

Alternatively or additionally to any of the embodiments above, the system may further include one or more additional openings are formed in the elongate catheter shaft that are in fluid communication with the one or more additional fluid delivery lumens.

Alternatively or additionally to any of the embodiments above, at least some of the one or more additional openings may be formed in a side wall of the elongate catheter shaft.

Alternatively or additionally to any of the embodiments above, at least some of the one or more additional openings may be side-facing openings.

Alternatively or additionally to any of the embodiments above, at least some of the one or more additional openings may be end-facing openings.

Another example may be found in an ultrasonic catheter system. The ultrasonic catheter system includes a multi-lumen catheter shaft having a central lumen and a plurality of fluid delivery lumens, and an ultrasound catheter core that is disposed within the central lumen, the ultrasound catheter core including one or more ultrasound transducers including a proximal-most ultrasound transducer. The central lumen is configured to allow for a cooling media to pass therethrough when the ultrasound catheter core is disposed within the central lumen. The plurality of fluid delivery lumens include a first fluid delivery lumen that extends along the multi-lumen catheter shaft to a fluid delivery lumen distal end that is disposed proximal of the proximal-most ultrasound transducer. A non-circular opening is formed in the multi-lumen catheter shaft at the fluid delivery lumen distal end, the non-circular opening being in fluid communication with the first fluid delivery lumen.

Alternatively or additionally to any of the embodiments above, the non-circular opening may be formed in a side wall of the multi-lumen catheter shaft.

Alternatively or additionally to any of the embodiments above, the non-circular opening may be a side-facing opening.

Alternatively or additionally to any of the embodiments above, the non-circular opening may be an end-facing opening.

Alternatively or additionally to any of the embodiments above, the non-circular opening may have an oval shape.

Another example may be found in a method for delivering a drug to a treatment site using an ultrasonic catheter system that includes a multi-lumen catheter shaft having a central lumen and a fluid delivery lumen, the fluid delivery lumen terminating at a distal end of the multi-lumen catheter shaft, and an ultrasound catheter core disposable within the central lumen, the ultrasound catheter core including a proximal-most ultrasound transducer. The method includes advancing the multi-lumen catheter shaft to a treatment site and advancing the ultrasound catheter core through the central lumen until the ultrasound catheter core reaches the distal end of the multi-lumen catheter shaft. The multi-lumen catheter shaft is withdrawn proximally such that the distal end of the multi-lumen catheter shaft is proximal of the proximal-most ultrasound transducer. A drug solution is passed through the fluid delivery lumen and the ultrasound catheter core is activated.

Alternatively or additionally to any of the embodiments above, the drug solution may include a plurality of microbubbles, phase shift nanodroplets, nanobubbles or any cavitation nuclei.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic illustration of certain features of an illustrative ultrasonic catheter;

FIG. 2 is a cross-sectional view taken along line 2-2 of FIG. 1;

FIG. 3 is a schematic illustration of an illustrative elongate inner core configured to be positioned within the central lumen of the catheter shown in FIG. 2;

FIG. 4 is a cross-sectional view taken along line 4-4 of FIG. 3;

FIG. 5 is a schematic wiring diagram illustrating a technique for electrically connecting five groups of ultrasound radiating members to form an ultrasound assembly;

FIG. 6 is a schematic wiring diagram illustrating a technique for electrically connecting one of the groups of FIG. 5;

FIG. 7A is a schematic illustration of the ultrasound assembly of FIG. 5 housed within the inner core of FIG. 4;

FIG. 7B is a cross-sectional view taken along line 7B-7B of FIG. 7A;

FIG. 7C is a cross-sectional view taken along line 7C-7C of FIG. 7A;

FIG. 7D is a side view of an ultrasound assembly center wire twisted into a helical configuration;

FIG. 8 illustrates the energy delivery section of the inner core of FIG. 4 positioned within the energy delivery section of the tubular body of FIG. 1;

FIG. 9 is a schematic view of an illustrative energy delivery portion of the illustrative ultrasonic catheter of FIG. 8;

FIG. 10 is a cross-sectional view taken along line 10-10 of FIG. 9;

FIG. 11 is a cross-sectional view taken along line 11-11 of FIG. 9;

FIG. 12 is a schematic view of an illustrative energy delivery portion of the illustrative ultrasonic catheter of FIG. 1;

FIG. 13 is a cross-sectional view taken along line 13-13 of FIG. 12;

FIG. 14 is a cross-sectional view taken along line 14-14 of FIG. 12;

FIG. 15 is a schematic view of an illustrative energy delivery portion of the illustrative ultrasonic catheter of FIG. 1; and

FIG. 16 is an end view of the illustrative energy delivery portion of FIG. 15.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

As used herein, the term “ultrasonic energy” is used broadly, includes its ordinary meaning, and further includes mechanical energy transferred through pressure or compression waves with a frequency greater than about 20 kHz. Ultrasonic energy waves have a frequency between about 500 kHz and about 20 MHz in one example embodiment, between about 1 MHz and about 3 MHz in another example embodiment, of about 3 MHz in another example embodiment, and of about 2 MHz in another example embodiment. As used herein, the term “catheter” is used broadly, includes its ordinary meaning, and further includes an elongate flexible tube configured to be inserted into the body of a patient, such as into a body part, cavity, duct or vessel. As used herein, the term “therapeutic compound” is used broadly, includes its ordinary meaning, and encompasses drugs, medicaments, dissolution compounds, genetic materials, and other substances capable of effecting physiological functions. A mixture comprising such substances is encompassed within this definition of “therapeutic compound”. As used herein, the term “end” is used broadly, includes its ordinary meaning, and further encompasses a region generally, such that “proximal end” includes “proximal region”, and “distal end” includes “distal region”.

As expounded herein, ultrasonic energy is often used to enhance the delivery and/or effect of a therapeutic compound. For example, in the context of treating vascular occlusions, ultrasonic energy has been shown to increase enzyme mediated thrombolysis by enhancing the delivery of thrombolytic agents into a thrombus, where such agents lyse the thrombus by degrading the fibrin that forms the thrombus. The thrombolytic activity of the agent is enhanced in the presence of ultrasonic energy in the thrombus. However, it should be appreciated that the invention should not be limited to the mechanism by which the ultrasound enhances treatment unless otherwise stated. In other applications, ultrasonic energy has also been shown to enhance transfection of gene-based drugs into cells, and augment transfer of chemotherapeutic drugs into tumor cells. Ultrasonic energy delivered from within a patient's body has been found to be capable of producing non-thermal effects that increase biological tissue permeability to therapeutic compounds by up to or greater than an order of magnitude.

Use of an ultrasound catheter to deliver ultrasonic energy and a therapeutic compound directly to the treatment site mediates or overcomes many of the disadvantages associated with systemic drug delivery, such as low efficiency, high therapeutic compound use rates, and significant side effects caused by high doses. Local therapeutic compound delivery has been found to be particularly advantageous in the context of thrombolytic therapy, chemotherapy, radiation therapy, and gene therapy, as well as in applications calling for the delivery of proteins and/or therapeutic humanized antibodies. However, it should be appreciated that in certain arrangements the ultrasound catheter can also be used in combination with systemic drug delivery instead or in addition to local drug delivery. In addition, local drug delivery can be accomplished through the use of a separate device (e.g., catheter).

As will be described below, the ultrasound catheter can include one or more ultrasound radiating members positioned therein. Such ultrasound radiating members can include a transducer (e.g., a PZT transducer), which is configured to convert electrical energy into ultrasonic energy. In such embodiments, the PZT transducer is excited by specific electrical parameters (herein “power parameters” that cause it to vibrate in a way that generates ultrasonic energy).

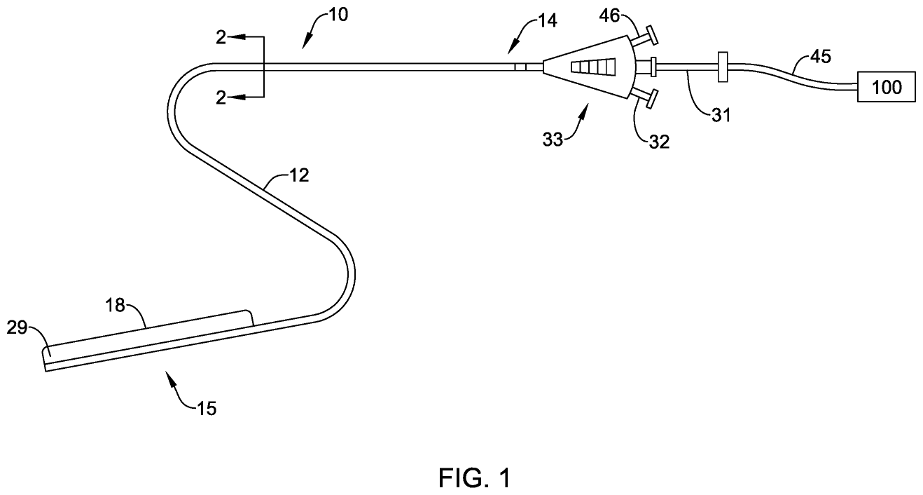

With reference to the illustrated embodiments, FIG. 1 illustrates an ultrasonic catheter 10 configured for use in a patient's vasculature. For example, in certain applications the ultrasonic catheter 10 is used to treat long segment peripheral arterial occlusions, such as those in the vascular system of the leg, while in other applications the ultrasonic catheter 10 is used to treat occlusions in the small vessels of the neurovasculature or other portions of the body (e.g., other distal portions of the vascular system). Thus, the dimensions of the catheter 10 may be adjusted based on the particular application for which the catheter 10 is to be used.

The ultrasonic catheter 10 generally includes a multi-component, elongate flexible tubular body 12 having a proximal region 14 and a distal region 15. The tubular body 12 includes a flexible energy delivery section 18 located in the distal region 15 of the catheter 10. The tubular body 12 and other components of the catheter 10 are manufactured in accordance with a variety of techniques. Suitable materials and dimensions are selected based on the natural and anatomical dimensions of the treatment site and on the desired percutaneous access site.

For example, in an embodiment the proximal region 14 of the tubular body 12 may include a material that has sufficient flexibility, kink resistance, rigidity and structural support to push the energy delivery section 18 through the patient's vasculature to a treatment site. Examples of such materials include, but are not limited to, extruded polytetrafluoroethylene (“PTFE”), polyethylenes (“PE”), polyamides and other similar materials. In certain embodiments, the proximal region 14 of the tubular body 12 may be reinforced by braiding, mesh or other constructions to provide increased kink resistance and pushability. For example, in certain embodiments nickel titanium or stainless steel wires may be placed along or incorporated into the tubular body 12 to reduce kinking.

In some instances, the energy delivery section 18 of the tubular body 12 may be formed of a material that (a) is thinner than the material forming the proximal region 14 of the tubular body 12, or (b) has a greater acoustic transparency than the material forming the proximal region 14 of the tubular body 12. Thinner materials generally have greater acoustic transparency than thicker materials. Suitable materials for the energy delivery section 18 include, but are not limited to, high or low density polyethylenes, urethanes, nylons, and the like. In some embodiments, the energy delivery section 18 is formed from the same material or a material of the same thickness as the proximal region 14.

One or more fluid delivery lumens may be incorporated into the tubular body 12. For example, in one embodiment a central lumen passes through the tubular body 12. The central lumen extends through the length of the tubular body 12, and is coupled to a distal exit port 29 and a proximal access port 31. The proximal access port 31 forms part of a hub 33, which is attached to the proximal region 14 of the catheter 10. In some cases, the hub 33 may include a cooling fluid fitting 46, which is hydraulically connected to a lumen within the tubular body 12. In some cases, the hub 33 may also include a therapeutic compound inlet port 32, which is hydraulically connected to a lumen within the tubular body 12. In some cases, the therapeutic compound inlet port 32 may also be hydraulically coupled to a source of therapeutic compound via a hub such as a Luer fitting.

The catheter 10 is configured to have one or more ultrasound radiating members positioned therein. For example, in certain embodiments an ultrasound radiating member may be fixed within the energy delivery section 18 of the tubular body, while in other embodiments a plurality of ultrasound radiating members are fixed to an assembly that is passed into the central lumen. In either case, the one or more ultrasound radiating members are electrically coupled to a control system 100 via a cable 45. In one embodiment, the outer surface of the energy delivery section 18 can include a cavitation promoting surface configured to enhance/promote cavitation at the treatment site. In some cases, a cavitation promoting surface is a textured surface that can retain small pockets of air when submerged. The small pockets of air can server as a source for microbubbles or nanobubbles, thereby reducing the threshold for cavitation in an ultrasound field. In some cases, the outer surface of the energy delivery section 18 may be coated with a coating that includes components that will lower the cavitation threshold. As an example, the surface may be hydrophobic and textured in a way so that the textured surface presents a lower cavitation threshold than the surrounding bulk fluid. This can enhance the therapeutic effect of the ultrasound.

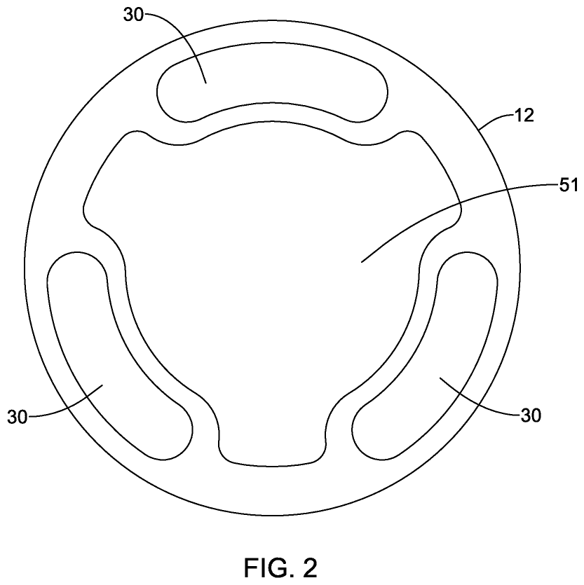

With reference to FIGS. 2-10, an exemplary arrangement of the energy delivery section 18 and other portions of the catheter 10 described above is shown. This arrangement is particularly well-suited for treatment of peripheral vascular occlusions.

FIG. 2 illustrates a cross section of the tubular body 12 taken along line 2-2 of FIG. 1. As shown in FIG. 2, three fluid delivery lumens 30 may be incorporated into the tubular body 12. In other embodiments, more or fewer fluid delivery lumens can be incorporated into the tubular body 12. The tubular body 12 may include a hollow central lumen 51 passing through the tubular body 12. The cross-section of the tubular body 12, as illustrated in FIG. 2, may be substantially constant along most of the length of the catheter 10. Thus, in such embodiments, substantially the same cross-section is present in both the proximal region 14 and the distal region 15 of the catheter 10. In some cases, the cross-section may vary within the energy delivery section 18, as will be discussed subsequently.

In certain embodiments, the central lumen 51 has a minimum diameter greater than about 0.030 inches. In another embodiment, the central lumen 51 has a minimum diameter greater than about 0.037 inches. In one preferred embodiment, the fluid delivery lumens 30 have dimensions of about 0.026 inches wide by about 0.0075 inches high, although other dimensions may be used in other applications.

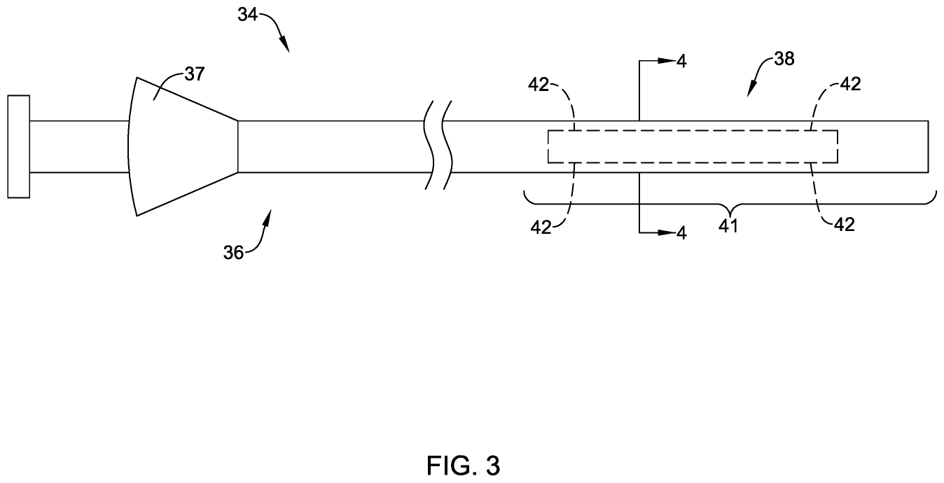

As described above, the central lumen 51 may extend through the length of the tubular body 12. As illustrated in FIG. 1, the central lumen 51 includes a distal exit port 29 and a proximal access port 31. The proximal access port 31 forms part of the hub 33, which is attached to the proximal region 14 of the catheter 10. The central lumen 51 may be configured to receive an elongate inner core 34 of which an embodiment is illustrated in FIG. 3. In some cases, the elongate inner core 34 includes a proximal region 36 and a distal region 38. A proximal hub 37 is fitted on the inner core 34 at one end of the proximal region 36. One or more ultrasound radiating members are positioned within an inner core energy delivery section 41 located within the distal region 38. The ultrasound radiating members 40 form an ultrasound assembly 42, which will be described in detail below.

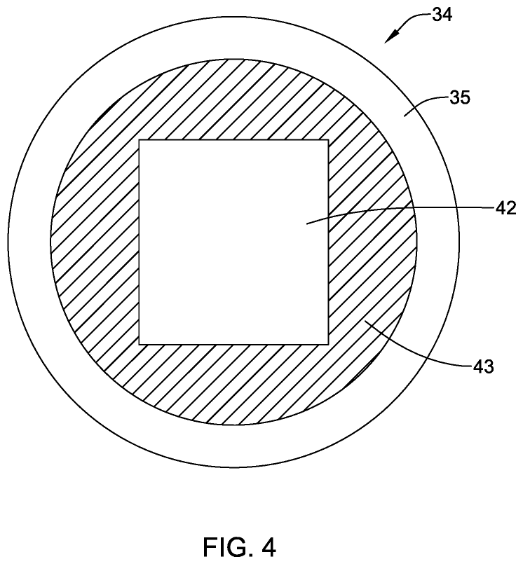

As shown in the cross-section illustrated in FIG. 4, which is taken along the line 4-4 of FIG. 3, the inner core 34 may have a cylindrical shape, with an outer diameter that permits the inner core 34 to be inserted into the central lumen 51 of the tubular body 12 via the proximal access port 31. Suitable outer diameters of the inner core 34 include, but are not limited to, about 0.010 inches to about 0.100 inches. In another embodiment, the outer diameter of the inner core 34 is between about 0.020 inches and about 0.080 inches. In yet another embodiment, the inner core 34 has an outer diameter of about 0.035 inches.

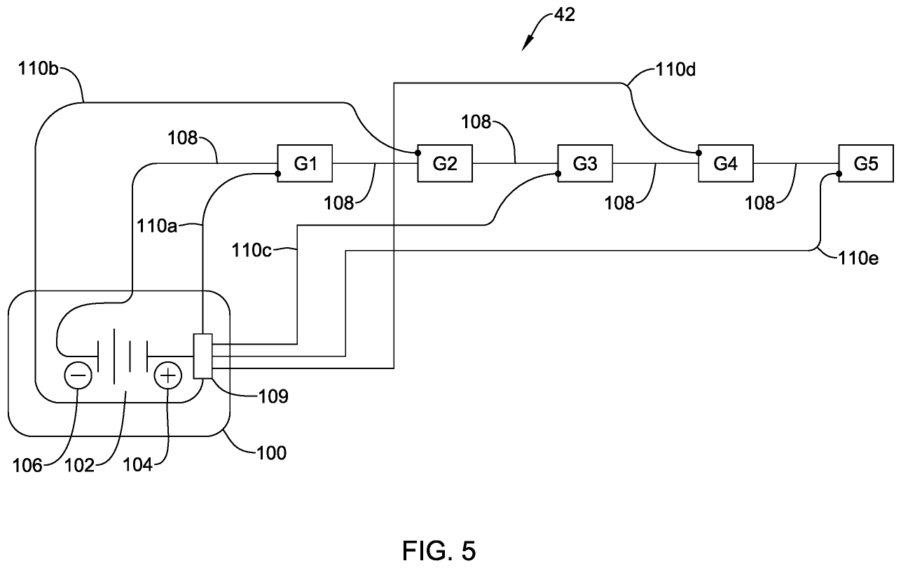

Still referring to FIG. 4, the inner core 34 may include a cylindrical outer body 35 that houses the ultrasound assembly 42. The ultrasound assembly 42 includes wiring and ultrasound radiating members, described in greater detail in FIGS. 5 through 7D, such that the ultrasound assembly 42 is capable of radiating ultrasonic energy from the energy delivery section 41 of the inner core 34. The ultrasound assembly 42 is electrically connected to the hub 33, where the inner core 34 can be connected to a control system 100 via cable 45 (illustrated in FIG. 1). In some cases, an electrically insulating potting material 43 fills the inner core 34, surrounding the ultrasound assembly 42, thus preventing movement of the ultrasound assembly 42 with respect to the outer body 35. In one embodiment, the thickness of the outer body 35 is between about 0.0002 inches and 0.010 inches. In another embodiment, the thickness of the outer body 35 is between about 0.0002 inches and 0.005 inches. In yet another embodiment, the thickness of the outer body 35 is about 0.0005 inches.

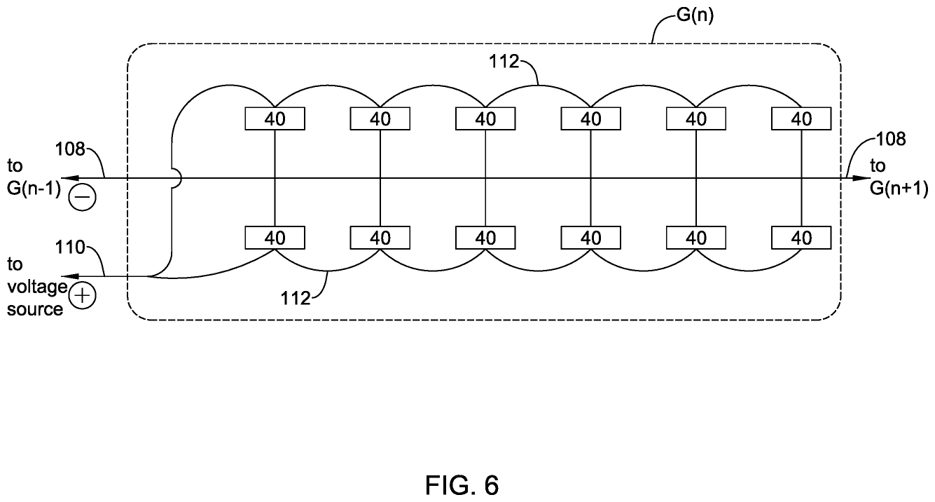

In some embodiments, the ultrasound assembly 42 includes a plurality of ultrasound radiating members 40 that are divided into one or more groups. For example, FIGS. 5 and 6 are schematic wiring diagrams illustrating a technique for connecting five groups of ultrasound radiating members 40 to form the ultrasound assembly 42. The ultrasound assembly 42 includes a set of transducer drivers 109, which includes a transducer driver that drives each of the five groups G1, G2, G3, G4, G5 of ultrasound radiating members 40 via electrical connections 110a, 110b, 110c, 110d and 110e, respectively. The five groups G1, G2, G3, G4, G5 of ultrasound radiating members 40 are also electrically connected to the control system 100. In some cases, a single amplifier is used, with a MUX to drive each of the individual groups, for example.

As used herein, the terms “ultrasonic energy”, “ultrasound” and “ultrasonic” are broad terms, having their ordinary meanings, and further refer to, without limitation, mechanical energy transferred through longitudinal pressure or compression waves. Ultrasonic energy can be emitted as continuous or pulsed waves, depending on the requirements of a particular application. Additionally, ultrasonic energy can be emitted in waveforms having various shapes, such as sinusoidal waves, triangle waves, square waves, or other wave forms. Ultrasonic energy includes sound waves. In certain embodiments, the ultrasonic energy has a frequency between about 20 kHz and about 20 MHz. For example, in one embodiment, the waves have a frequency between about 500 kHz and about 20 MHz. In another embodiment, the waves have a frequency between about 1 MHz and about 3 MHz. In yet another embodiment, the waves have a frequency of about 2 MHz. The average acoustic power for each ultrasound radiating member 40 is between about 0.01 watts and 300 watts. In some embodiments, the average acoustic power for each ultrasound radiating member 40 is about 0.2 watts and about 2.5 watts. In an embodiment, the average acoustic power for each ultrasound radiating member 40 is about 0.27 watts.

As used herein, the term “ultrasound radiating member” refers to any apparatus capable of producing ultrasonic energy. For example, in one embodiment, an ultrasound radiating member comprises an ultrasonic transducer, which converts electrical energy into ultrasonic energy. A suitable example of an ultrasonic transducer for generating ultrasonic energy from electrical energy includes, but is not limited to, piezoelectric ceramic oscillators. Piezoelectric ceramics may include a crystalline material, such as quartz, that changes shape when an electrical current is applied to the material. This change in shape, made oscillatory by an oscillating driving signal, creates ultrasonic sound waves. In other embodiments, ultrasonic energy can be generated by an ultrasonic transducer that is remote from the ultrasound radiating member, and the ultrasonic energy can be transmitted, via, for example, a wire that is coupled to the ultrasound radiating member.

Still referring to FIG. 5, the control circuitry 100 may include, among other things, a voltage source 102. The voltage source 102 includes a positive terminal 104 and a negative terminal 106. The negative terminal 106 is connected to common wire 108, which connects the five groups G1-G5 of ultrasound radiating members 40 in series. The positive terminal 104 is connected to a plurality of lead wires 110a, 110b, 110c, 110d and 110e, which connect to one of the five groups G1-G5 of ultrasound radiating members 40, respectively. Thus, under this configuration, each of the five groups G1-G5, one of which is illustrated in FIG. 6, is connected to the positive terminal 104 via one of the lead wires 110a, 110b, 110c, 110d and 110e, and to the negative terminal 106 via the common wire 108. The control circuitry can be configured as part of the control system 100 and can include circuits, control routines, controllers etc. configured to vary one or more power parameters used to drive ultrasound radiating members 40.

Referring now to FIG. 6, each group G1-G5 includes a plurality of ultrasound radiating members 40. Each of the ultrasound radiating members 40 is electrically connected to the common wire 108 and to the lead wire 310 via one of two positive contact wires 112. Thus, when wired as illustrated, a constant voltage difference will be applied to each ultrasound radiating member 40 in the group. Although the group illustrated in FIG. 6 includes twelve ultrasound radiating members 40, one of ordinary skill in the art will recognize that more or fewer ultrasound radiating members 40 can be included in the group. Likewise, more or fewer than five groups can be included within the ultrasound assembly 42 illustrated in FIG. 5.

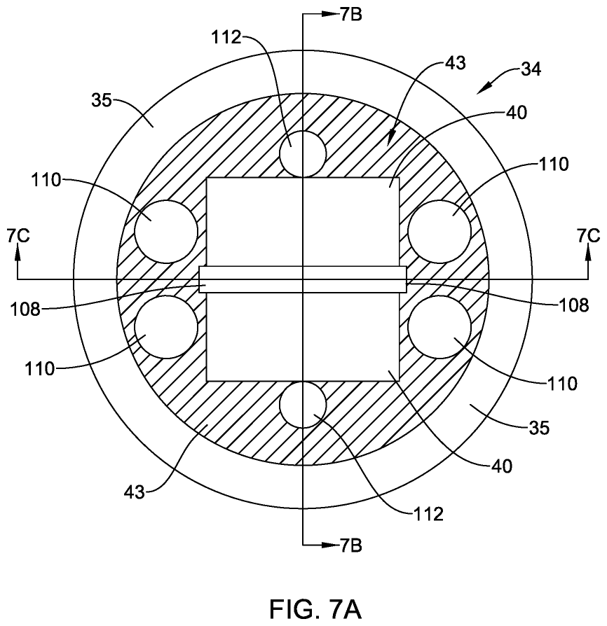

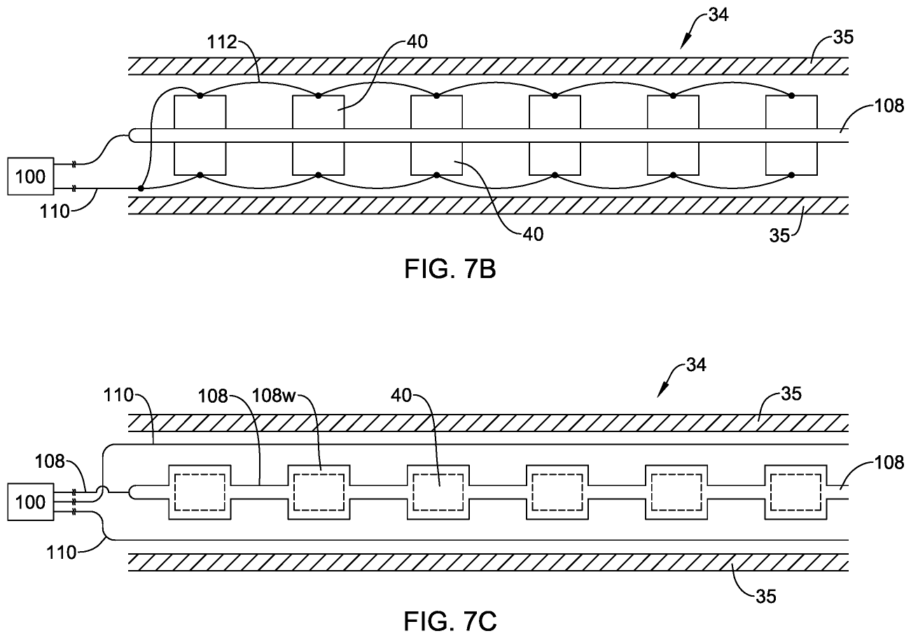

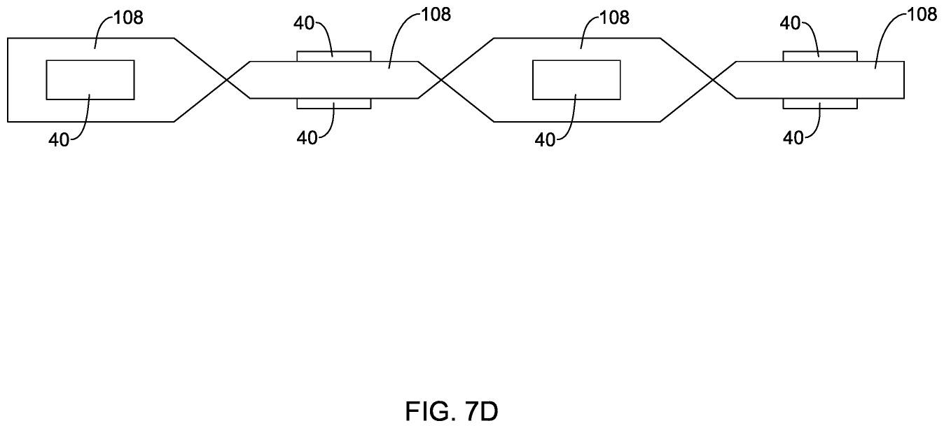

FIG. 7A illustrates one preferred technique for arranging the components of the ultrasound assembly 42 (as schematically illustrated in FIG. 5) into the inner core 34 (as schematically illustrated in FIG. 4). FIG. 7A is a cross-sectional view of the ultrasound assembly 42 taken within group G1 in FIG. 5, as indicated by the presence of four lead wires 110. For example, if a cross-sectional view of the ultrasound assembly 42 was taken within group G4 in FIG. 5, only one lead wire 310 would be present (that is, the one lead wire connecting group G5).

Referring still to FIG. 7A, the common wire 108 includes an elongate, flat piece of electrically conductive material in electrical contact with a pair of ultrasound radiating members 40. Each of the ultrasound radiating members 40 is also in electrical contact with a positive contact wire 312. Because the common wire 108 is connected to the negative terminal 106, and the positive contact wire 312 is connected to the positive terminal 104, a voltage difference can be created across each ultrasound radiating member 40. Lead wires 110 may be separated from the other components of the ultrasound assembly 42, thus preventing interference with the operation of the ultrasound radiating members 40 as described above. For example, in one embodiment, the inner core 34 may be filled with an insulating potting material 43, thus deterring unwanted electrical contact between the various components of the ultrasound assembly 42.

FIGS. 7B and 7C illustrate cross sectional views of the inner core 34 of FIG. 7A taken along lines 7B-7B and 7C-7C, respectively. As illustrated in FIG. 7B, the ultrasound radiating members 40 are mounted in pairs along the common wire 108. The ultrasound radiating members 40 are connected by positive contact wires 112, such that substantially the same voltage is applied to each ultrasound radiating member 40. As illustrated in FIG. 7C, the common wire 108 may include wide regions 108W upon which the ultrasound radiating members 40 can be mounted, thus reducing the likelihood that the paired ultrasound radiating members 40 will short together. In certain embodiments, outside the wide regions 108W, the common wire 108 may have a more conventional, rounded wire shape.

In an embodiment, such as illustrated in FIG. 7D, the common wire 108 may be twisted to form a helical shape before being fixed within the inner core 34. In such embodiments, the ultrasound radiating members 40 are oriented in a plurality of radial directions, thus enhancing the radial uniformity of the resulting ultrasonic energy field.

One of ordinary skill in the art will recognize that the wiring arrangement described above can be modified to allow each group G1, G2, G3, G4, G5 to be independently powered. Specifically, by providing a separate power source within the control system 100 for each group, each group can be individually turned on or off, or can be driven with an individualized power. This provides the advantage of allowing the delivery of ultrasonic energy to be “turned off” in regions of the treatment site where treatment is complete, thus preventing deleterious or unnecessary ultrasonic energy to be applied to the patient.

The embodiments described above, and illustrated in FIGS. 5 through 7, illustrate a plurality of ultrasound radiating members grouped spatially. That is, in such embodiments, all of the ultrasound radiating members within a certain group are positioned adjacent to each other, such that when a single group is activated, ultrasonic energy is delivered at a specific length of the ultrasound assembly. However, in some embodiments, the ultrasound radiating members of a certain group may be spaced apart from each other, such that the ultrasound radiating members within a certain group are not positioned adjacent to each other. In such embodiments, when a single group is activated, ultrasonic energy can be delivered from a larger, spaced apart portion of the energy delivery section. Such modified embodiments may be advantageous in applications wherein it is desired to deliver a less focused, more diffuse ultrasonic energy field to the treatment site.

In some embodiments, the ultrasound radiating members 40 may include rectangular lead zirconate titanate (“PZT”) ultrasound transducers that have dimensions of about 0.017 inches by about 0.010 inches by about 0.080 inches. In other embodiments, other configurations may be used. For example, disc-shaped ultrasound radiating members 40 can be used in other embodiments. In an embodiment, the common wire 108 includes copper, and is about 0.005 inches thick, although other electrically conductive materials and other dimensions can be used in other embodiments. Lead wires 110 may be 36 gauge electrical conductors, for example, while positive contact wires 112 may be 42 gauge electrical conductors. However, one of ordinary skill in the art will recognize that other wire gauges can be used in other embodiments.

As described above, suitable frequencies for the ultrasound radiating member 40 include, but are not limited to, from about 20 kHz to about 20 MHz. In one embodiment, the frequency is between about 500 kHz and 20 MHz, and in another embodiment 1 MHz and 3 MHz. In yet another embodiment, the ultrasound radiating members 40 are operated with a frequency of about 2 MHz.

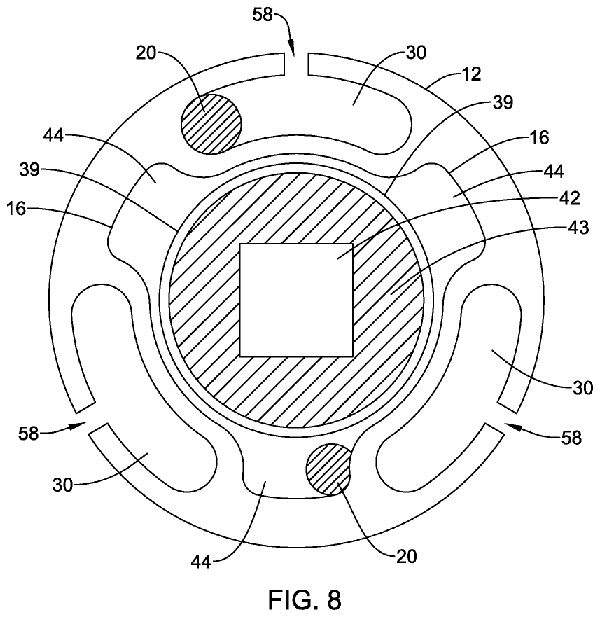

FIG. 8 illustrates the inner core 34 positioned within the tubular body 12. Details of the ultrasound assembly 42, provided in FIG. 7A, are omitted for clarity. As described above, the inner core 34 can be slid within the central lumen 51 of the tubular body 12, thereby allowing the inner core energy delivery section 41 to be positioned within the tubular body energy delivery section 18. For example, in an embodiment, the materials including the inner core energy delivery section 41, the tubular body energy delivery section 18, and the potting material 43 may all be materials having a similar acoustic impedance, thereby minimizing ultrasonic energy losses across material interfaces.

FIG. 8 further illustrates placement of fluid delivery ports 58 within the tubular body energy delivery section 18. As illustrated, holes or slits are formed from the fluid delivery lumen 30 through the tubular body 12, thereby permitting fluid flow from the fluid delivery lumen 30 to the treatment site. Thus, a source of therapeutic compound coupled to the inlet port 32 provides a hydraulic pressure which drives the therapeutic compound through the fluid delivery lumens 30 and out the fluid delivery ports 58.

By evenly spacing the fluid delivery lumens 30 around the circumference of the tubular body 12, as illustrated in FIG. 8, a substantially even flow of therapeutic compound around the circumference of the tubular body 12 can be achieved. In addition, the size, location and geometry of the fluid delivery ports 58 can be selected to provide uniform fluid flow from the fluid delivery ports 30 to the treatment site. For example, in one embodiment, fluid delivery ports closer to the proximal region of the energy delivery section 18 have smaller diameters then fluid delivery closer to the distal region of the energy delivery section 18, thereby allowing uniform delivery of fluid across the entire energy delivery section.

For example, in one embodiment in which the fluid delivery ports 58 have similar sizes along the length of the tubular body 12, the fluid delivery ports 58 have a diameter between about 0.0005 inches to about 0.0050 inches. In another embodiment in which the size of the fluid delivery ports 58 changes along the length of the tubular body 12, the fluid delivery ports 58 have a diameter between about 0.001 inches to about 0.005 inches in the proximal region of the energy delivery section 18, and between about 0.005 inches to 0.020 inches in the distal region of the energy delivery section 18. The increase in size between adjacent fluid delivery ports 58 depends on the material comprising the tubular body 12, and on the size of the fluid delivery lumen 30. The fluid delivery ports 58 can be created in the tubular body 12 by punching, drilling, burning or ablating (such as with a laser), or by any other suitable method. Therapeutic compound flow along the length of the tubular body 12 can also be increased by increasing the density of the fluid delivery ports 58 toward the distal region 15 of the tubular body 12.

In the case of delivery of cavitation nuclei such as microbubbles, nanobubbles, microdroplets or nanodroplets, it can be beneficial to make the fluid delivery ports 58 large enough so that the cavitation nuclei are not subject to excessive pressure or shear stresses as the cavitation nuclei traverse the fluid delivery lumens 30 and exit the fluid delivery ports 58. It should be appreciated that it may be desirable to provide non-uniform fluid flow from the fluid delivery ports 58 to the treatment site. In such embodiments, the size, location and geometry of the fluid delivery ports 58 can be selected to provide such non-uniform fluid flow.

Referring still to FIG. 8, placement of the inner core 34 within the tubular body 12 further defines cooling fluid lumens 44. Cooling fluid lumens 44 are formed between an outer surface 39 of the inner core 34 and an inner surface 16 of the tubular body 12. In some embodiments, a cooling fluid may be introduced through the proximal access port 31 such that cooling fluid flow is produced through cooling fluid lumens 44 and out the distal exit port 29 (see FIG. 1). In some cases, the cooling fluid lumens 44 may be evenly spaced around the circumference of the tubular body 12 (that is, at about 120° increments for a three-lumen configuration), thereby providing uniform cooling fluid flow over the inner core 34. Such a configuration is useful for removing unwanted thermal energy at the treatment site. The flow rate of the cooling fluid and the power to the ultrasound assembly 42 may be adjusted to maintain the temperature of the distal region 15 of the catheter 10 within a desired range. In some cases, the desired temperature range may be between 28° C. and 52° C. In some cases, the desired temperature range may be between 28° C. and 45° C. In some cases, the desired temperature range may be between 28° C. and 43° C.

In an embodiment, the inner core 34 can be rotated or moved within the tubular body 12. Specifically, movement of the inner core 34 can be accomplished by maneuvering the proximal hub 37 while holding the hub 33 stationary. The inner core outer body 35 is at least partially constructed from a material that provides enough structural support to permit movement of the inner core 34 within the tubular body 12 without kinking of the tubular body 12. Additionally, the inner core outer body 35 may include a material having the ability to transmit torque. Suitable materials for the inner core outer body 35 include, but are not limited to, polyimides, polyesters, polyurethanes, thermoplastic elastomers and braided polyimides.

In an embodiment, the fluid delivery lumens 30 and the cooling fluid lumens 44 are open at the distal end of the tubular body 12, thereby allowing the therapeutic compound and the cooling fluid to pass into the patient's vasculature at the distal exit port. Or, if desired, the fluid delivery lumens 30 can be selectively occluded at the distal end of the tubular body 12, thereby providing additional hydraulic pressure to drive the therapeutic compound out of the fluid delivery ports 58. In either configuration, the inner core 34 can be prevented from passing through the distal exit port by making the inner core 34 with a length that is less than the length of the tubular body. In other embodiments, a protrusion is formed on the internal side of the tubular body in the distal region 15, thereby preventing the inner core 34 from passing through the distal exit port.

In still other embodiments, the catheter 10 may further include an occlusion device (not shown) positioned at the distal exit port 29. The occlusion device may have a reduced inner diameter that can accommodate a guidewire, but that is less than the inner diameter of the central lumen 51. Thus, the inner core 34 is prevented from extending through the occlusion device and out the distal exit port 29. For example, suitable inner diameters for the occlusion device include, but are not limited to, about 0.005 inches to about 0.050 inches. In other embodiments, the occlusion device has a closed end, thus preventing cooling fluid from leaving the catheter 10, and instead recirculating to the proximal region 14 of the tubular body 12. These and other cooling fluid flow configurations permit the power provided to the ultrasound assembly 42 to be increased in proportion to the cooling fluid flow rate. Additionally, certain cooling fluid flow configurations can reduce exposure of the patient's body to cooling fluids.

In certain embodiments, as illustrated in FIG. 8, the tubular body 12 may further include one or more temperature sensors 20, that may be located within the energy delivery section 18. In such embodiments, the proximal region 14 of the tubular body 12 includes a temperature sensor lead which can be incorporated into cable 45 (illustrated in FIG. 1). Suitable temperature sensors include, but are not limited to, temperature sensing diodes, thermistors, thermocouples, resistance temperature detectors (“RTDs”) and fiber optic temperature sensors which use thermalchromic liquid crystals. Suitable temperature sensor 20 geometries include, but are not limited to, a point, a patch or a stripe. The temperature sensors 20 can be positioned within one or more of the fluid delivery lumens 30 (as illustrated), and/or within one or more of the cooling fluid lumens 44.

The ultrasound radiating members may be operated in a pulsed mode. For example, in one embodiment, the time average electrical power supplied to the ultrasound radiating members 40 is between about 0.001 watts and about 5 watts and can be between about 0.05 watts and about 3 watts. In some embodiments, the time average electrical power over treatment time is about 0.45 watts or 1.2 watts. The duty cycle is between about 0.01% and about 90% and can be between about 0.1% and about 50%. In certain embodiments, the duty ratio is about 7.5%, 15% or a variation between 1% and 30%. The pulse averaged electrical power for each ultrasound radiating member 40 can be between about 0.01 watts and about 20 watts and can be between about 0.1 watts and 20 watts. In certain embodiments, the pulse averaged electrical power is about 4 watts, 8 watts, 16 watts, or a variation of 0.5 to 8 watts. As described above, the amplitude, pulse width, pulse repetition frequency, peak negative acoustic pressure or any combination of these parameters can be constant or varied during each pulse or over a set of pulses. In a non-linear application of acoustic parameters the above ranges can change significantly. Accordingly, the overall time average electrical power over treatment time may stay the same but not real-time average power.

In one embodiment, the pulse repetition rate may be between about 1 Hz and about 2 kHz and more can be between about 1 Hz and about 50 Hz. In another embodiment, the pulse repetition rate is about 30 Hz, or a variation of about 10 Hz to about 40 Hz. The pulse duration or width can be between about 0.5 millisecond and about 50 milliseconds and can be between about 0.1 millisecond and about 25 milliseconds. In some embodiments, the pulse duration is about 2.5 milliseconds, 5 or a variation of 1 to 8 milliseconds. In addition, the peak negative acoustic pressure can be between about 0.1 to about 50 MPa or in another embodiment between about 0.5 to about 2.0 MPa.

In one embodiment, the transducers are operated at an average power of about 0.6 watts, a duty cycle of about 7.5%, a pulse repetition rate of about 30 Hz, a pulse average electrical power of about 8 watts and a pulse duration of about 2.5 milliseconds.

The ultrasound radiating member used with the electrical parameters described herein may have an acoustic efficiency greater than about 50% and can be greater than about 75%. The ultrasound radiating member can be formed a variety of shapes, such as, cylindrical (solid or hollow), flat, bar, triangular, and the like. The length of the ultrasound radiating member may be between about 0.1 cm and about 0.7 cm. The thickness or diameter of the ultrasound radiating members may be between about 0.02 cm and about 0.5 cm.

In certain embodiments, the therapeutic compound delivered to the treatment site includes a plurality of microbubbles having, for example, a gas formed therein. Exemplary gases that are usable to form the microbubbles include, but are not limited to, air, oxygen, carbon dioxide, perfluorocarbon gases and inert gases.

In some embodiments, the microbubble-therapeutic compound can include about 10⁴ microbubbles per milliliter of liquid to about 10¹⁰ microbubbles per millimeter of liquid, or from about 10⁶ to about 10⁹ microbubbles per milliliter of liquid. In some embodiments the microbubbles in the microbubble-therapeutic compound have a diameter of between about 0.1 μm and about 30 μm. In some embodiments, the microbubbles have a diameter of about 0.1 to about 10 μm, about 0.2 to about 10 μm, about 0.5 to about 10 μm, about 0.5 to about 5 μm, or about 1 μm. In some embodiments, the microbubbles have a diameter of less than or equal to about 10 μm, about 5 μm, or about 2.5 μm. Other parameters can be used in other embodiments.

In some embodiments, the efficacy of the therapeutic compound is enhanced by the presence of the microbubbles contained therein. In some embodiments, the microbubbles can act as a nucleus for cavitation, and thus allow cavitation to be induced at lower levels of peak rarefaction acoustic pressure. Therefore, a reduced amount of peak rarefaction acoustic pressure can be delivered to the treatment site without reducing the efficacy of the treatment. Reducing the amount of ultrasonic pressure delivered to the treatment site reduces risks associated with overheating the treatment site, and, in certain embodiments, also reduces the time required to treat a vessel. In some embodiments, cavitation also promotes more effective diffusion and penetration of the therapeutic compound into surrounding tissues, such as the vessel wall and/or the clot material. Furthermore, in some embodiments, the mechanical agitation caused by cavitation of the microbubbles is effective in mechanically breaking up clot material.

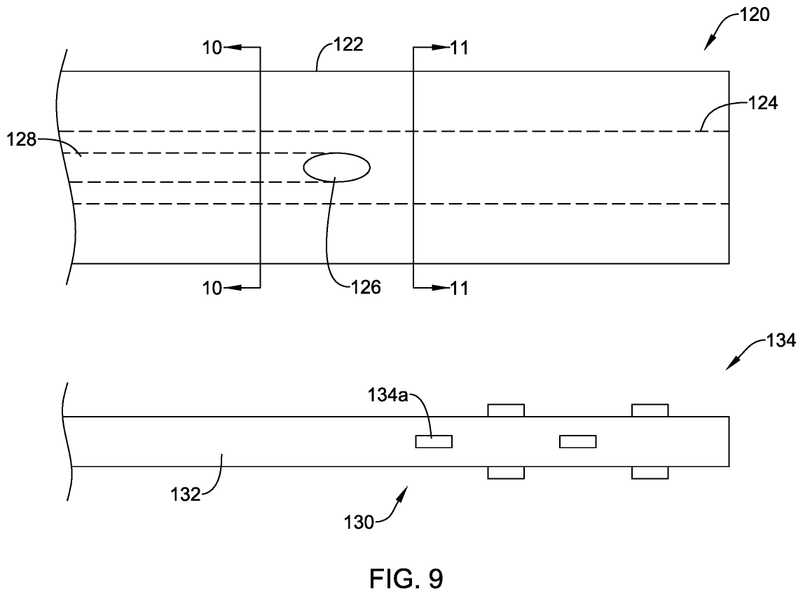

In some cases, when infusion a solution that includes microbubbles, there may be a desire to adapt the ultrasonic catheter 10 in a way that results in the solution having exited the ultrasonic catheter 10 proximal to any of the ultrasound transducers. In some cases, the microbubbles may be subject to bursting when the microbubbles are subjected to ultrasonic energy while the solution (and hence the microbubbles) remain within a fluid delivery lumen. FIGS. 9 through 16 provide illustrative but non-limiting examples of modified energy delivery sections (such as the energy delivery section 18) that may provide for microbubble solution delivery or delivery of any cavitation nuclei that allows the solution to exit the catheter prior to being exposed to ultrasonic energy.

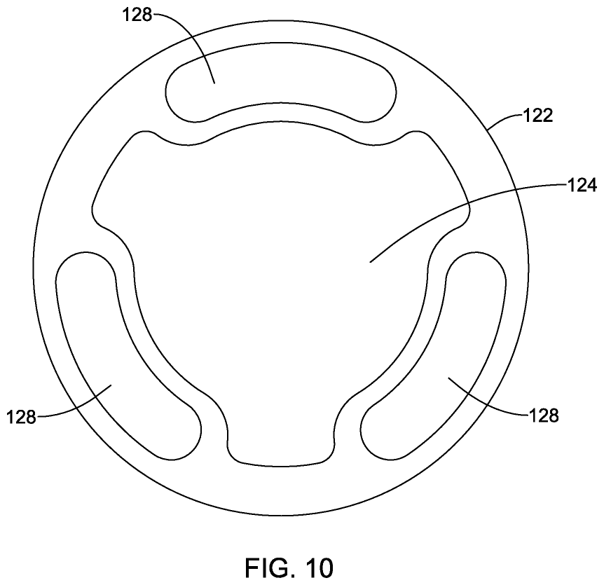

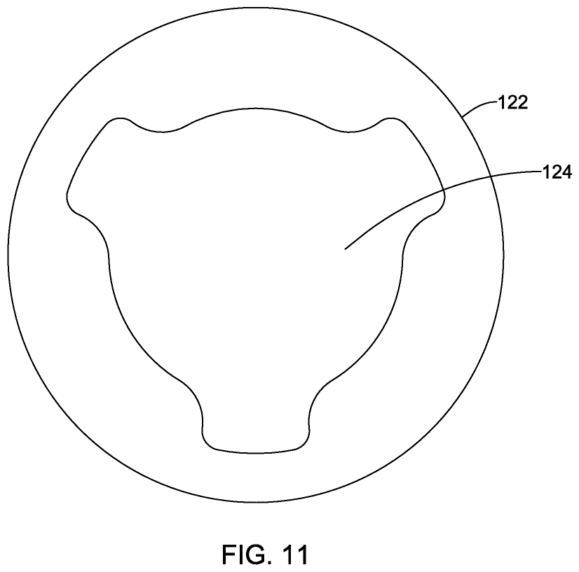

FIG. 9 is a schematic view of an illustrative energy delivery section 120. FIGS. 10 and 11 are cross-sectional views taken along the line 10-10 and 11-11, respectively, of FIG. 9. The illustrative energy delivery section 120 may be considered as being an example of the energy delivery section 18, and may utilize any of the materials and material properties described with respect to the energy delivery section 18. The energy delivery section 120 includes a tubular body 122. In some cases, the tubular body 122 defines one or more lumens extending within the tubular body 122. As an example, the tubular body 122 defines a central lumen 124 (shown in phantom), which may be considered as corresponding to the central lumen 51. In some cases, the central lumen 124 extends all the way through the energy delivery section 120, and may align with a central lumen such as the central lumen 51 extending through the rest of the ultrasonic catheter 10.

The tubular body 122 includes a fluid opening 126 that extends through a wall of the tubular body 122. The fluid opening 126 may be considered as being a side-facing opening as the fluid opening 126 extends through a side-wall of the tubular body 122. The fluid opening 126 may be any desired shape. In some cases, the fluid opening 126 may have a non-circular shape, such as but not limited to an ovoid or oval shape. In some instances, the fluid opening 126 may be larger in one direction than in another direction. For example, the fluid opening 126 may have a length that is twice its width. Other ratios are also contemplated. The fluid opening 126 is in fluid communication with a fluid delivery lumen 128, shown in phantom. While only a single fluid delivery lumen 128 is shown, it will be appreciated that the tubular body 122 may include several fluid delivery lumens 128 arranged parallel with each other and the central lumen 124, and may be radially offset from the central lumen 124. As an example, there may be a total of three fluid delivery lumens 128, each with a non-circular cross-sectional shape, as shown for example in FIG. 10.

FIG. 9 also shows an ultrasound core 130 including an elongate member 132 that is adapted to fit within the central lumen 124, but is shown outside of the energy delivery section 120 for clarity. The ultrasound core 130 includes a number of ultrasound transducers 134 that are secured relative to the elongate member 132, and may be considered as being arranged in pairs. The ultrasound core 130 may have any desired number of ultrasound transducers 134, arrange in any manner. The ultrasound transducers 134 include a proximal-most ultrasound transducer 134a. With the ultrasound core 130 shown in parallel with the tubular body 122, it can be seen that the proximal-most ultrasound transducer 134a is arranged such that the proximal-most ultrasound transducer 134a is distal of the fluid opening 126. As a result, any drug solution eluting from the fluid opening 126 will be outside of the energy delivery section 120 before the drug solution encounters any ultrasonic energy being produced by the ultrasound transducers 134, including the proximal-most ultrasound transducer 134a. The cavitation nuclei are delivered upstream from the ultrasound field and are allowed to flow through the ultrasound field outside of the fluid delivery lumens. The fluid delivery lumens 128 terminate proximal of the proximal-most ultrasound transducer 134a, as can be seen in FIG. 11. FIG. 11 shows the central lumen 124 continuing, but not the fluid delivery lumens 128.

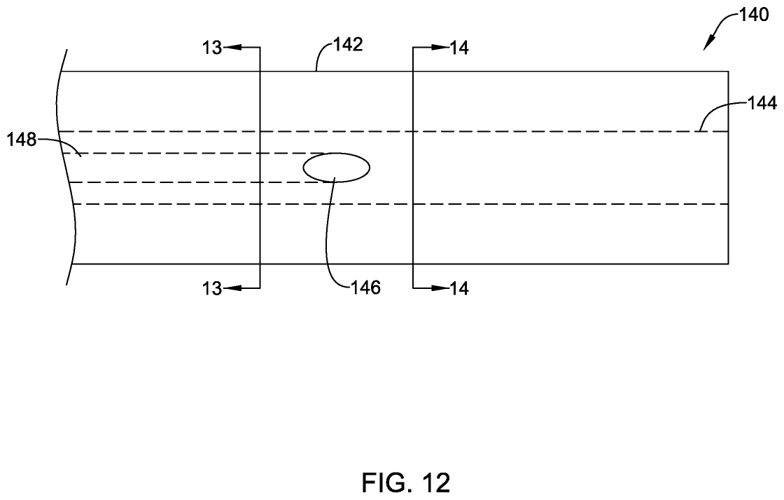

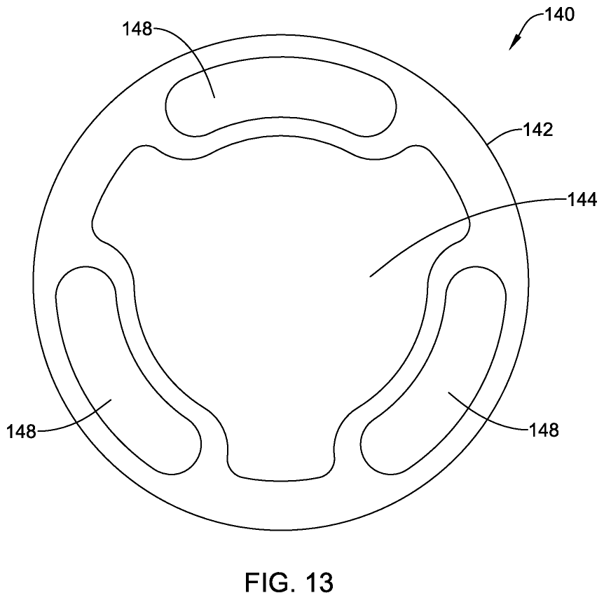

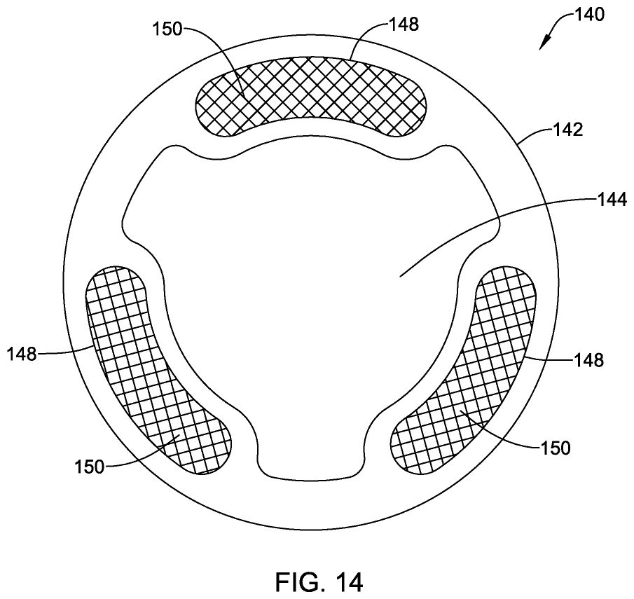

FIG. 12 is a schematic view of an illustrative energy delivery section 140. FIGS. 13 and 14 are cross-sectional views taken along the line 13-13 and 14-14, respectively, of FIG. 12. The illustrative energy delivery section 140 may be considered as being an example of the energy delivery section 18, and may utilize any of the materials and material properties described with respect to the energy delivery section 18. The energy delivery section 140 includes a tubular body 142. In some cases, the tubular body 142 defines one or more lumens extending within the tubular body 142. As an example, the tubular body 142 defines a central lumen 144 (shown in phantom), which may be considered as being equivalent to the central lumen 51. In some cases, the central lumen 144 extends all the way through the energy delivery section 140, and may align with a central lumen such as the central lumen 51 extending through the rest of the ultrasonic catheter 10. The central lumen 144 may be considered as accommodating an ultrasound core such as the ultrasound core 130 (not shown in FIG. 12).

The tubular body 142 includes a fluid opening 146 that extends through a wall of the tubular body 142. The fluid opening 146 may be considered as being a side-facing opening as the fluid opening 146 extends through the side-wall of the tubular body 142. The fluid opening 146 is in fluid communication with a fluid delivery lumen 148, shown in phantom. The fluid opening 146 may be any desired shape. In some cases, the fluid opening 146 may have a non-circular shape, such as but not limited to an ovoid or oval shape. In some instances, the fluid opening 146 may be larger in one direction than in another direction. For example, the fluid opening 146 may have a length that is twice its width. Other ratios are also contemplated. While only a single fluid delivery lumen 148 is shown, it will be appreciated that the tubular body 142 may include several fluid delivery lumens 148 arranged parallel with each other and the central lumen 144, and may be radially offset from the central lumen 144. As an example, there may be a total of three fluid delivery lumens 148, each with a non-circular cross-sectional shape, as shown for example in FIG. 13.

Any solution eluting from the fluid opening 146 will be outside of the energy delivery section 140 before the solution encounters any ultrasonic energy being produced by the ultrasound transducers 134, including the proximal-most ultrasound transducer 134a. The cavitation nuclei are delivered upstream from the ultrasound field and are allowed to flow through the ultrasound field outside of the fluid delivery lumens. In some cases, the fluid delivery lumens 148 as defined by the tubular body 142 may continue distally, but may be blocked. As can be seen in FIG. 14, the central lumen 144 continues but the fluid delivery lumens 148 have been blocked by a material 150. Any of a variety of different materials may be used as the material 150, including epoxy, polyurethane or any flexible polymer.

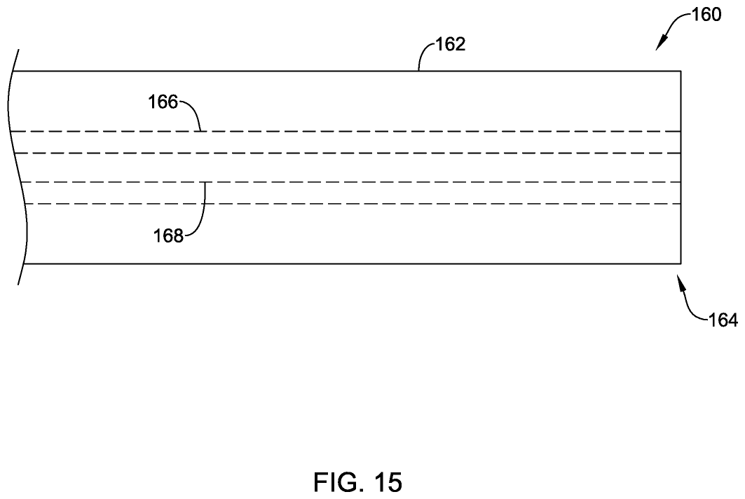

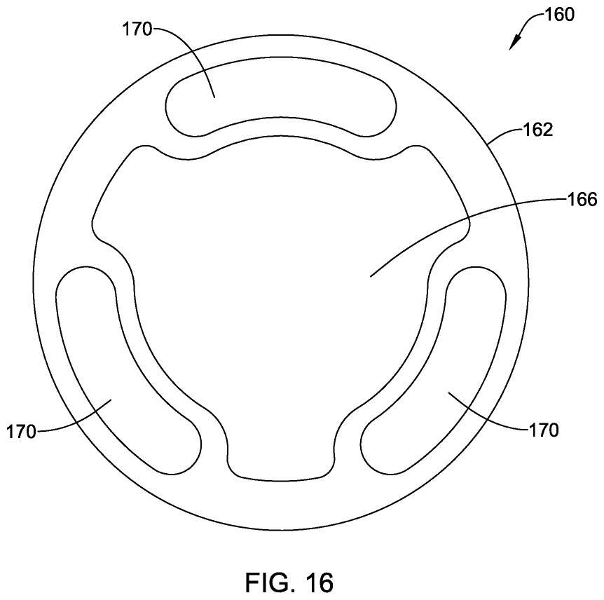

FIG. 15 is a schematic view of an illustrative energy delivery section 160. FIG. 16 is an end view thereof. The illustrative energy delivery section 160 may be considered as being an example of the energy delivery section 18, and may utilize any of the materials and material properties described with respect to the energy delivery section 18. The energy delivery section 160 includes a tubular body 162 extending to a distal end 164. In some cases, the tubular body 162 defines one or more lumens extending within the tubular body 162. As an example, the tubular body 162 defines a central lumen 166 (shown in phantom). In some cases, the central lumen 166 extends all the way through the energy delivery section 160, and may align with a central lumen such as the central lumen 51 extending through the rest of the ultrasonic catheter 10. The central lumen 164 may be considered as accommodating an ultrasound core such as the ultrasound core 130 (not shown in FIG. 15). The tubular body 162 defines several fluid delivery lumens 168 (a total of three are shown).

As seen in FIG. 16, the fluid delivery lumens 168 terminate in fluid openings 170 that are formed in the distal end 164 of the tubular body 162. The fluid openings 170 may be considered as being end-facing openings, rather than side-facing openings, since the fluid openings 170 are located at the distal end 164 of the tubular body 162. In use, the catheter including the energy delivery section 160 would be advanced to a treatment site. The ultrasound core 130 (FIG. 9) would be advanced through the catheter and into the energy delivery section 160. Prior to eluting any drug, or activating the ultrasound transducers 134, the catheter including the energy delivery section 160 would be withdrawn proximally a short distance, such that the fluid openings 170 are proximal of the proximal-most ultrasound transducer 134a. As a result, any drug solution eluting from the fluid openings 170 will be outside of the energy delivery section 160 before the drug solution encounters any ultrasonic energy being produced by the ultrasound transducers 134, including the proximal-most ultrasound transducer 134a. The cavitation nuclei are delivered upstream from the ultrasound field and are allowed to flow through the ultrasound field outside of the fluid delivery lumens.

The materials that can be used for the various components of the devices described herein may include those commonly associated with medical devices. The devices and components thereof described herein may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the devices described herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the devices described herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the devices described herein to achieve the same result.

In some embodiments, a degree of Magnetic Resonance Imaging (MRI) compatibility is imparted into the devices described herein. For example, the devices described herein, or portions thereof, may be made of a material that does not substantially distort the image and create substantial artifacts (e.g., gaps in the image). Certain ferromagnetic materials, for example, may not be suitable because they may create artifacts in an MRI image. The devices described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for treating a vascular region, the system comprising:
an elongate catheter shaft having a distal end region;
wherein an inner lumen is formed in the elongate catheter shaft;
wherein a fluid delivery lumen is formed in the elongate catheter shaft and positioned adjacent to the inner lumen;
a treatment core disposed within the inner lumen, the treatment core including one or more ultrasound transducers disposed adjacent to the distal end region of the elongate catheter shaft, the one or more ultrasound transducers including a proximal-most ultrasound transducer; and
wherein the fluid delivery lumen is adapted such that fluid passing through the fluid delivery lumen exits the fluid delivery lumen at a position proximal to the proximal-most ultrasound transducer.

2. The system of claim 1, wherein the inner lumen has a non-circular cross-sectional shape.

3. The system of claim 1, wherein the inner lumen has a central treatment region and one or more coolant regions disposed about the central treatment region.

4. The system of claim 1, wherein the fluid delivery lumen includes an opening that is formed in a side wall of the elongate catheter shaft.

5. The system of claim 4, wherein the opening is a side-facing opening.

6. The system of claim 4, wherein the opening is end-facing.

7. The system of claim 4, wherein the opening has a non-circular shape.

8. The system of claim 4, wherein the opening has an oval shape.

9. The system of claim 1, further comprising one or more additional fluid delivery lumens that are formed in the elongate catheter shaft and are positioned adjacent to the inner lumen.

10. The system of claim 4, further comprising one or more additional openings are formed in the elongate catheter shaft that are in fluid communication with the one or more additional fluid delivery lumens.

11. The system of claim 10, wherein at least some of the one or more additional openings are formed in a side wall of the elongate catheter shaft.

12. The system of claim 10, wherein at least some of the one or more additional openings are side-facing openings.

13. The system of claim 10, wherein at least some of the one or more additional openings are end-facing.

14. An ultrasonic catheter system, comprising:
a multi-lumen catheter shaft having a central lumen and a plurality of fluid delivery lumens;
an ultrasound catheter core disposed within the central lumen, the ultrasound catheter core including one or more ultrasound transducers including a proximal-most ultrasound transducer;
wherein the central lumen is configured to allow for a cooling media to pass therethrough when the ultrasound catheter core is disposed within the central lumen;
wherein the plurality of fluid delivery lumens include a first fluid delivery lumen that extends along the multi-lumen catheter shaft to a fluid delivery lumen distal end that is disposed proximal of the proximal-most ultrasound transducer; and
wherein a non-circular opening is formed in the multi-lumen catheter shaft at the fluid delivery lumen distal end, the non-circular opening being in fluid communication with the first fluid delivery lumen.

15. The ultrasonic catheter system of claim 14, wherein the non-circular opening is formed in a side wall of the multi-lumen catheter shaft.

16. The ultrasonic catheter system of claim 14, wherein the non-circular opening is a side-facing opening.

17. The ultrasonic catheter system of claim 14, wherein the non-circular opening is end-facing.

18. The ultrasonic catheter system of claim 14, wherein the non-circular opening has an oval shape.

19. A method for delivering a drug to a treatment site using an ultrasonic catheter system that includes a multi-lumen catheter shaft having a central lumen and a fluid delivery lumen, the fluid delivery lumen terminating at a distal end of the multi-lumen catheter shaft, and an ultrasound catheter core disposable within the central lumen, the ultrasound catheter core including a proximal-most ultrasound transducer, the method comprising:
advancing the multi-lumen catheter shaft to a treatment site;
advancing the ultrasound catheter core through the central lumen until the ultrasound catheter core reaches the distal end of the multi-lumen catheter shaft;
withdrawing the multi-lumen catheter shaft proximally such that the distal end of the multi-lumen catheter shaft is proximal of the proximal-most ultrasound transducer;
passing a drug solution through the fluid delivery lumen; and
activating the ultrasound catheter core.

20. The method of claim 19, wherein the drug solution comprises a plurality of microbubbles, nanobubbles, phase shift nanodroplets, or any cavitation nuclei.
